(19) United States
(12) Patent Application Publication
(10) Pub. No.: US 2024/0337646 A1
(43) Pub. Date: Oct. 10, 2024

(54) COMPOSITIONS AND METHODS FOR TREATING MELANOMA

(71) Applicant: Johns Hopkins University, Baltimore, MD (US)

(72) Inventors: Xinzhong Dong, Baltimore, MD (US); Naina Gour, Baltimore, MD (US)

(21) Appl. No.: 18/293,692

(22) PCT Filed: Jul. 29, 2022
(86) PCT No.: PCT/US22/38958

§ 371 Date: Jan. 30, 2024

Related U.S. Application Data
(60) Provisional application No. 63/227,984, filed on Jul. 30, 2021.

(51) Int. Cl. G01N 33/50; A61K 38/17; A61K 39/00; C07K 16/28

(52) U.S. Cl. CPC G01N 33/5011; A61K 38/177; C07K 16/28; A61K 2039/505; G01N 2333/726

(57) ABSTRACT

Compositions and methods are provided that include use of one or more MrgprX4 antagonists to treat melanoma.

### CROSS-REFERENCE TO RELATED APPLICATION

This application claims the benefit of U.S. Provisional Application 63/227,984 filed Jul. 30, 2021, which is incorporated by reference herein in its entirety.

### BACKGROUND

G protein coupled receptor mediated disorders including chronic itch, inflammation disorders, cancer, autoimmunity, skin disorders, and adverse drug reactions cause suffering. Much is unknown regarding the pathology of G protein coupled mediated disorders. Amongst these disorders, there is a need for novel cancer therapeutics as patients with this disease often develop resistance to standard therapies.

Melanoma, the cancer arising from melanocytes, is one of the deadliest cancers because of its high invasion potential. The incidence for melanoma has been on the rise in the past decades posing a serious burden on healthcare.

### SUMMARY

We now provide new treatments for melanoma which include use of one or more MrgprX4 antagonist compounds.

In one aspect, methods of treating melanoma in a subject are provided which include administering an effective amount of an MrgprX4 antagonist compound, thereby treating the melanoma.

In a further aspect, methods are provided diagnosing the presence of melanoma in a subject, which may include determining the levels of expression of MrgprX4 in a sample of the subject; and correlating the levels of MrgprX4 expression in the sample with the presence of melanoma.

In such methods, the presence (or increased expression) of MrgprX4 is indicative of the presence of melanoma in the subject from whom the sample is taken. Typically, these methods are performed in vitro, although in vivo methods are not necessarily excluded. The sample can be a tissue sample (e.g., a skin biopsy), blood sample or a serum sample.

The levels of MrgprX4 expression may vary with different stages of the disease. Accordingly, in methods that determine the presence of melanoma, a further step may be inc [symbol] lates the levels of MrgprX4 expression to disease seve [symbol] (e.g., stage of melanoma), or disease progression.

Methods for screening and identifying agents that are useful to treat melanoma are also provided and may include contacting one or more cells expressing an MrgprX4 G protein coupled receptor with a candidate drug agent; and detecting a response of the one or more cells to thereby select the candidate drug agent for treatment of melanoma.

We have found that MrgprX4 expression at particularly high levels in melanoma samples as demonstrated in the examples which follow.

In some embodiments of the present methods and compostions, an MrgprX4 antagonist compound comprises an antibody or fragment thereof, a binding protein, a polypeptide, or any combination thereof. In some embodiments, an MrgprX4 antagonist compound comprises a small molecule. In some embodiments, an MrgprX4 antagonist compound comprises a nucleic acid molecule. In some embodiments, the nucleic acid molecule comprises double stranded ribonucleic acid (dsRNA), small hairpin RNA or short hairpin RNA (shRNA), or antisense RNA, or any portion thereof. In some embodiments of the present methods, an MrgprX4 antagonist compound is administered topically, orally, via inhalation, or via injection.

In certain preferred aspects, an MrgprX4 antagonist compound is a small molecule. Exemplary suitable MrgprX4 antagonists include xanthine (2,6-dihydroxy purine) compounds including 3-substituted xanthine compounds. Exemplary preferred xanthine compounds for use as an MrgprX4 antagonist compound as set forth herein are disclosed in WO2022/079245 (PCT/EP2021/078622), including compounds B-1 to B-58, C1-C10, D1-D3, E1-E3, F1, G1-G3 and J1-J3 as disclosed in WO2022/079245 (PCT/EP2021/078622) which is incorporated by reference herein in its entirety.

Additional MrgprX4 antagonist compounds for use in the present methods and compositions including MrgprX4 antagonist small molecules for use in the present method and compositions are disclosed in WO2020/198537 (PCT/US2020/025077), incorporated herein by reference in its entirety.

Also provided herein are pharmaceutical compositions for the treatment of melanoma, the composition comprising an effective amount an MrgprX4 antagonist. Further provided are kits that comprise 1) a pharmaceutical composition as disclosed herein and 2) written instructions for treating melanoma.

Also provided herein are methods for identifying an antagonist of MrgprX4 or comprising: contacting the isolated cell (e.g. an isolated cell comprising a recombinant nucleic acid that expresses mas-related G-protein coupled receptor member X4 (M [symbol] candidate antagonist, detecting activation of MrgprX4, [symbol] in activation of MrgprX4 relative to the activation of MrgprX4 in the absence of the compound (i.e. control) determines that the candidate compound is an antagonist. Preferably, a candidate antagonist decreases activation of MrgprX4 by at least 1, 2, 3, 4 or 5 percent relative to a test assay in the absence of the candidate antagonist (control), more preferably a candidate antagonist decreases activation of MrgprX4 in a test assay by at least 7, 10, 15, 20, 25, 30, 40 50 60, 70, 80, 90 or 100 percent relative to the same test assay in the absence of the candidate antagonist (control). Preferred test assays for assessing a candidate antagonist include an assay of Example 9 of WO2018/232316 where activation can be assessed by calcium imaging. Activation also can be assessed by inositol phosphate detection and β-arrestin recruitment.

As discussed, provided herein are methods for treating malignant transformations, particularly melanoma in a subject comprising identifying a subject suffering from or at risk of developing melanoma and administering to the subject an effective amount of a composition comprising an MrgprX4 antagonist, thereby treating or preventing the melanoma in a subject.

The subject is preferably a mammal in need of such treatment or prophylaxis, e.g., a subject that has been diagnosed with a melanoma or a predisposition thereto (e.g. prior diagnosis of melanoma). The mammal is any mammal, e.g., a human, a primate, a mouse, a rat. In a preferred embodiment, the mammal is a human.

The MrgprX4 inhibitors or antagonists may include but are not limited to nucleic acids, peptides, antibodies, or small molecules that bind to their specified target or the target's natural ligand and modulate the biological activity.

In some cases, the MrgprX4 antagonist comprises a small molecule. A small molecule is a compound that is less than 2000 Daltons in mass. The molecular mass of the small molecule is preferably less than 1000 Daltons, more preferably less than 600 Daltons, e.g., the compound is less than 500 Daltons, less than 400 Daltons, less than 300 Daltons, less than 200 Daltons, or less than 100 Daltons.

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. In certain aspects, typically small molecules are less than one kilodalton. [symbol] es, the MrgprX4 antagonist comprises a nucleic acid [symbol] example, ribonucleic acid (RNA) or deoxyribonucleic acid (DNA) inhibits the expression of MrgprX4 polypeptide, thereby inhibiting the activity of MrgprX4. In some cases, the nucleic acid comprises small interfering RNA (siRNA), RNA interference (RNAi), messenger RNA (mRNA), small hairpin RNA or short hairpin RNA (shRNA), double stranded ribonucleic acid (dsRNA), antisense RNA or microRNA, or any portion thereof. However, the skilled artisan could readily identify additional nucleic acids that inhibit/antagonize MrgprX4.

As discussed, an MrgprX4 antagonist can be an antibody, for example a monoclonal or polyclonal MrgprX4 antibody. For instance, MrgprX4 antibodies that may be employed as an antagonist include monoclonal or polyclonal antibodies, such as mouse, rabbit, primate (e.g. monkey) or humanized antibodies (e.g., commercially available Novus Biologicals rabbit polyclonal MrgprX4 antibody No. NLS2429; Abcam's rabbit polyclonal MrgprX4 antibody b97784; Abcam's rabbit polyclonal MrgrpX4 antibody ab 188740; and Thermo Fischer's anti-MrgprX4 polyclonal antibody PA5-3395). Fragments of such monoclonal antibodies also can be suitable antagonists or agonists, including fragments of the noted commercially available antibodies. Suitable and preferred antibody fragments for use as an MrgprX4 antagonist can be readily identified by the assays disclosed herein. Suitable fragments may contain a sequence that has at least 30, 40, 50, 60, 70, 80, 90 or 95 sequence identity with the corresponding antibody such as the noted commercially available antibodies. Such fragments may be the entire agent that is used an MrgprX4 antagonist or may be covalently linked to another sequence or other molecule, for instance to form a fusion molecule containing the antibody fragment sequence, or containing a sequence having a suitable sequence identify with the corresponding antibody such as the noted commercially available antibodies.

As also discussed herein, suitable and preferred MrgprX4 antagonists including small molecules, polypeptides, antibodies and antibody fragments, and nucleic acids can be readily identified including by the assays disclosed herein.

MrgprX4 antagonists are also disclosed in WO2018/232316 and US2021/0032213, both incorporated herein by reference.

A variety of administration routes are available. For example, the antagonist or agonist is administered topically, orally, via inhalation, or via injection.

The effective amount of an MrgprX4 antagonist is from for example 0.001 mg/kg to 250 mg/kg body weight, e.g., 0.001 mg/kg, 0.05 mg/kg 0.01 mg/kg, 0.05 mg/kg, 1 mg/kg, 5 mg/kg, 10 mg/kg, 25 mg/kg, 50 mg/kg, 75 mg/kg, 100 mg/kg, 125 mg/kg, 150 mg/kg, 175 mg/kg, 200 mg/kg, 225 mg/kg, or 250 mg/kg body weight. Ultimately, the attending ph [symbol] narian decides the appropriate amount and dosage regi [symbol] In some cases, an MrgprX4 antagonist is administered at least once per day, at least once per week, or at least once per month. The antagonist or agonist suitably may be administered for a duration of one day, one week, one month, two months, three months, six months, 9 months, or one year. In some cases, a MrgprX4 antagonist may be administered daily, e.g., every 24 hours. Alternatively, a MrgprX4 the antagonist is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Activation of MrgprX4 can be detected by identifying an increase in intracellular calcium relative to the level of intracellular calcium in the absence of the compound. In some cases, the level of intracellular calcium increases by at least 1%, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. Intracellular calcium concentration is determined utilizing the methods described herein or those available to the skilled artisan.

A candidate MrgprX4 antagonist can be screened to confirm that it counteracts or inhibits, decreases, or suppresses the biological activity of an MrgprX4 polypeptide.

A decrease in activation of MrgprX4 relative to the activation of MrgprX4 in the absence of the candidate antagonist determines that the candidate compound is an antagonist. Preferably, a candidate antagonist decreases activation of MrgprX4 by at least 1, 2, 3, 4 or 5 percent relative to a test assay in the absence of the candidate antagonist (control), more preferably a candidate antagonist decreases activation of MrgprX4 in a test assay by at least 7, 10, 15, 20, 25, 30, 40 50 60, 70, 80, 90 or 100 percent relative to the same test assay in the absence of the candidate antagonist (control). Preferred test assays for assessing a candidate antagonist include an assay of Example 9 of WO2018/232316, where activation can be assessed by calcium imaging or inositol phosphate or β-arrestin recruitment based detection.

Also provided are methods for identifying an agonist of MrgprX4 comprising: contacting the isolated cell described herein (e.g. an isolated cell comprising a recombinant nucleic acid that expresses mas-related G-protein coupled receptor MrgprX4) with a compound that induces a pseudo-allergic-type reaction, contacting the isolated cell described herein with a candidate agonist, detecting activation of MrgprX4, wherein an increase in activation of MrgprX4 relative to the activation of MrgprX4 in the absence of the candidate agonist determines that the candidate compound is an agonist. Preferably, a selected car [symbol] decreases activation of MrgprX4 by at least 1, 2, 3, 4 o [symbol] to a test assay in the absence of the candidate antagonist (control), more preferably a selected candidate agonist decreases activation of MrgprX4 in a test assay by at least 7, 10, 15, 20, 25, 30, 40 50 60, 70, 80, 90 or 100 percent relative to the same test assay in the absence of the candidate antagonist (control). Preferred test assays for assessing a candidate agonist include an assay of Example 9 of WO2018/232316, where activation can be assessed by calcium imaging or inositol phosphate detection or β-arrestin recruitment based assay.

The composition described herein are administered via oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

### Definitions

Unless defined otherwise, all technical and scientific terms used herein have the meaning commonly understood by a person skilled in the art to which this invention belongs. The following references provide one of skill with a general definition of many of the terms used in this invention: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Antibodies and fragments thereof described herein include, but are not limited to, polyclonal, monoclonal, chimeric, dAb (domain antibody), single chain, Fab, Fab′ and F(ab′)2 fragments, Fv, scFvs. A fragment of an antibody possess the immunological activity of its respective antibody. In some embodiments, a fragment of an antibody contains 1500 or less, 1250 of less, 1000 or less, 900 or less, 800 or less, 700 or less, 600 or less, 500 or less, 400 or less, 300 or less, 200 or less amino acids. For example, a protein or peptide inhibitor contains 1500 or less, 1250 of less, 1000 or less, 900 or less, 800 or less, 700 or less, 600 or less, 500 or less, 400 or less, 300 or less, 200 or less, 100 or less, 80 or less, 70 or less, 60 or less, 50 or less, 40 or less, 30 or less, 25 or less, 20 or less, 10 or less amino acids. For example, a nucleic acid inhibitor of the invention contains 400 or less, 300 or less, 200 or less [symbol] 00 or less, 90 or less, 80 or less, 70 or less, 60 or less, [symbol] 35 or less, 30 or less, 28 or less, 26 or less, 24 or less, 22 or less, 20 or less, 18 or less, 16 or less, 14 or less, 12 or less, 10 or less nucleotides.

General methods in molecular and cellular biochemistry can be found in such standard textbooks as Molecular Cloning: A Laboratory Manual, 4th Ed. (Sambrook et al., Cold Spring Harbor Laboratory Press 2012); Short Protocols in Molecular Biology, 5th Ed. (Ausubel et al. eds., John Wiley & Sons 2002); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to [symbol] ne covalent disulfide bond, while the two H chains are [symbol] by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an ant [symbol] gen, but exhibit various effector functions, such as par [symbol] antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally, the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., [symbol] 22:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody nat [symbol] by a human. However, as used herein, human antibo [symbol] residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H} 1, C_{H} 2 and C_{H} 3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8 (10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the [symbol] region is also the part recognized by Fc receptors (F [symbol] in types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, HuM2e antibody specifically binds to M2e if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or flu [symbol] ated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist antibody” is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

As used herein, the term “epitope” includes any protein determinant capable of specific binding to an immunoglobulin, an scFv, or a T-cell receptor. Epitopic determinants consist of chemically active surface groupings of molecules such as amino acids or sugar side chains and have specific three dimensional structural characteristics, as well as specific cha [symbol] ics. For example, antibodies may be raised against N- [symbol] terminal peptides of a polypeptide, linear or non-linear peptide sequences of a protein, as well as epitopes that comprise amino acids of a first antigen and those of a second antigen.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (Nature 361:186-87 (1993)). The ratio of K_{off}/Kₒₙ enables the cancellation of all parameters not related to affinity, and is equal to the dissociation constant K_{d}. Davies et al. (1990) Annual Rev Biochem 59:439-473). An antibody may be said to specifically bind to an antigen or epitope described herein when the equilibrium binding constant (K_{d}) is ≤1 μM, preferably ≤100 nM, more preferably ≤10 nM, more preferably ≤1 nM, and most preferably ≤100 pM to about 1 pM, as measured by assays such as radioligand binding assays or similar assays known to those skilled in the art.

The invention also comprises polypeptides and nucleic acid fragments, so long as they exhibit the desired biological activity (i.e., antagonize MrgprX4) of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

Polynucleotides, polypeptides, or other agents are purified and/or isolated. Specifically, as used herein, an “isolated” or “purified” nucleic acid molecule, polynucleotide, polypeptide, or pro [symbol] ally free of other cellular material, or culture medium [symbol] recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic complementary DNA (cDNA), a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

The term “vector” is used to refer to a carrier nucleic acid molecule into which a het [symbol] ic acid sequence can be inserted for introduction into [symbol] e replicated and expressed. The term further denotes certain biological vehicles useful for the same purpose, e.g. viral vectors and phage—both these infectious agents are capable of introducing a heterelogous nucleic acid sequence

An “expression vector” is a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector.

By a “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components and derivatives thereof.

The term “pharmaceutical composition” is meant any composition, which contains at least one therapeutically or biologically active agent and is suitable for administration to the patient. Any of these formulations can be prepared by well-known and accepted methods of the art. See, for example, Remington: The Science and Practice of Pharmacy, 20th edition, (ed. A. R. Gennaro), Mack Publishing Co., Easton, Pa., 2000.

By “G protein-coupled receptors (GPCR)” is meant a protein receptor that senses molecules outside a cell and activates, inside the cell, signal transduction pathways and, ultimately, cellular responses. GPCRs are called seven-transmembrane receptors because they pass through the cell membrane seven times.

By “agonist” is meant a chemical that binds to a receptor and activates the receptor to pr [symbol] al response. Whereas an agonist causes an action, an “ [symbol] the action of the agonist and an inverse agonist causes an action opposite to that of the agonist. As used herein, the terms “antagonist” and “inhibitor” are used interchangeably to refer to any molecule that counteracts or inhibits, decreases, or suppresses the biological activity of its target molecule. In some embodiments, an agonist is a “superagonist” when it induces or increases the biological activity of its target molecule (e.g., MrgprX4). In some embodiments, an antagonist is a “superantagonist” when it counteracts or inhibits, decreases, or suppresses the biological activity of its target molecule (e.g., MrgprX4). Suitable MrgprX4 antagonists include soluble receptors, peptide inhibitors, small molecule inhibitors, ligand fusions, and antibodies.

The term “administering,” as used herein, refers to any mode of transferring, delivering, introducing, or transporting an MrgprX4 antagonist, for example, to a subject in need of treatment for a disease or condition. Such modes include, but are not limited to, oral, topical, intravenous, intraperitoneal, intr

By “alteration” is meant a change (increase or decrease) in the activity of polypeptide, e.g. MrgprX4, as detected by standard methods known in the art such as those described herein. As used herein, an alteration includes a 10% or more change in expression levels or activity of a gene or polypeptide, preferably a 25% change, more preferably a 40% change, and most preferably a 50% or greater change in activity of polypeptide.

As used herein an “alteration” also includes a 2-fold or more change in expression levels or activity of a gene or polypeptide, for example, 5-fold, 10-fold, 20-fold, 30-fold, 40-fold, 50-fold, 100-fold, 500-fold, 1000-fold or more.

By “ameliorate” is meant decrease, suppress, attenuate, diminish, arrest, or stabilize the development or progression of a disease such as, for example, cancer, including melanoma. By “amplify” is meant to increase the number of copies of a molecule. In one example, the polymerase chain reaction (PCR) is used to amplify nucleic acids.

By “binding” is meant having a physicochemical affinity for a molecule. Binding is measured by any of the methods of the invention, e.g., a drug/compound with a receptor expressed on a cell.

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that wh [symbol] at excludes prior art embodiments.

“Detect” refers to identifying, either directly or indirectly, the presence, absence, or amount of MrgprX4 inhibition or activation of a signal transduction pathway to be detected.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” [symbol] ‘MrgprX4 antagonist compound” and “MrgprX4 antag [symbol] interchangeably herein as understood.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1B: FIG. 1A (top panel) is a gross photo of mouse tail mass. FIG. 1B (bottom panel) is a microscopic photo of mouse tail mass.

FIGS. 2A-2B: FIG. 2A (left panel) is a gross photo of mouse ear mass. FIG. 2B (right panel) is a microscopic photo of mouse tail mass.

FIG. 3 is a photomicrograph of mouse tail mass.

FIG. 4 shows MRGPRX4 expression in control skin versus melanoma.

FIG. 5 shows MRGPRX4 expression in various cancers. Red highlighted is cutaneous melanoma.

FIG. 6A-6B shows images of inguinal lymph nodes (40× and 10× magnification) depicting metastasis.

FIG. 7 (includes FIGS. 7a-7e) and FIG. 8 (includes FIGS. 8a-8d) show results from Example 2 which follows.

FIG. 9 (includes FIGS. 9a-9e) shows results from Example 3 which follows.

FIG. 10 shows results from Example 4 which follows.

FIG. 11 (includes FIGS. 11a-11c) shows results from Example 5 which follows.

FIG. 12 shows results from Example 6 which follows.

FIG. 13 shows results of Example 7 which follows.

### DE CRIPTION

### MrgprX4

Mas-related G-protein coupled receptor member X4 is a protein that in humans is encoded by the MRGPRX4 gene. MRGPRX4 on sensory neurons is known to bind bile acid and mediate cholestatic itch (PMID: 31068464 & PMID: 31500698). Our novel data using mouse models and human melanocytes and melanoma cell lines highlights the role of MRGPRX4 as a driver of metastatic melanoma. See WO2018/232316.

### Treatment Methods

As discussed, methods are provided for treatment of a subject suffering or susceptible to melanoma.

In certain aspects, the melanoma to be treated can by any of all kinds of melanoma including for example melanoma of skin, eyes and/or meninges.

Thus, methods include identifying a patient as suffering from a melanoma (including melanoma of skin, eyes and/or meninges) and administering to the identified subject an effective of an MrgprX4 antagonist, for example an MrgprX4 antagonist that is a small molecule or antibody or antibody fragment.

In certain aspects, the melanoma to be treated is a cutaneous melanoma. Thus, methods include identifying a patient as suffering from a cutaneous melanoma and administering to the identified subject an effective of an MrgprX4 antagonist compound, for example an MrgprX4 antagonist compound that is a small molecule or antibody or antibody fragment.

In certain aspects, the melanoma to be treated is a metastatic melanoma. Thus, methods include identifying a patient as suffering from a metastatic melanoma and administering to the identified subject an effective of an MrgprX4 antagonist compound, for example an MrgprX4 antagonist compound that is a small molecule or antibody or antibody fragment.

In certain aspects, the melanoma is a metastatic malignant melanoma. Thus, methods include identifying a patient as suffering from a metastatic malignant melanoma and administering to the identified subject an effective of an MrgprX4 antagonist, for example an MrgprX4 antagonist compound that is a small molecule or antibody or antibody fragment. In some aspects, the melanoma is a stage IV melanoma. In some aspects, the subject or patient has distant metastases. In some aspects, the metastatic melanoma is stage M1a. In some aspects, the metastatic melanoma is stage M1b. In some embodiments, the me [symbol] a is stage M1c. In some embodiments, the metastatic [symbol] M1d. In some aspects, the subject or patient has a measurable disease.

In certain aspects, the subject or patient has not previously been treated for melanoma. In certain aspects, the subject or patient has not received prior cytotoxic chemotherapy or immunotherapy for metastatic malignant melanoma. In certain aspects, the subject or patient has not received prior adjuvant cytotoxic chemotherapy or immunotherapy.

In certain aspects, the patient or subject is a male. In certain aspects, the patient or subject is a female. In certain aspects, the patient or subject is less than 65 years of age. In certain aspects, the patient or subject is at least 65 years old (including at least about 70, 75 or 80 years old).

In certain aspects, the patient or subject has a melanoma tumor (e.g. biopsy sample) that exhibits overexpression of MrgprX4 relative to control tissue, for example at least about 2, 5, 10, 20, 50, or 100 times greater expression relative to a control sample.

In certain aspects, a composition comprising one or more MrgprX4 antagonist compounds as disclosed herein is used as monotherapy to treat melanoma.

In certain aspects, a further or second distinct therapy may be administered as subject in addition to administration of one or more one or more MrgprX4 antagonist compounds. For instance, the second distinct therapy may include one or more of treatment with one or more distinct inhibitors (for ex: Braf and MEK inhibitors: vemurafenib, dabrafenib, cobimetenib etc.), chemotherapy agents, immunotherapy (anti-PD-1 agents such as nivolumab and pembrolizumab, anti-CTLA-4 agents, etc.), surgery or radiation therapy.

In certain aspects, a composition comprising one or more MrgprX4 antagonist compounds is administered intravenously.

In certain aspects, a composition comprising one or more MrgprX4 antagonist compounds may be administered transdermally or topically.

### Pharmaceutical Compositions

In certain embodiments, the present invention provides for a pharmaceutical composition comprising one or more MrgprX4 antagonist compounds. The MrgprX4 antagonist(s) can be suitably formulated and introduced into a subject or the environment of a cell by any means recognized for such delivery.

Suitable MrgprX4 antagonists: In certain preferred aspects, an MrgprX4 antagonist compound is a small molecule. Exemplary suitable MrgprX4 antagonist compounds include xanthine (2,6-dihydroxy purine) compounds including 3-substituted xanthine compounds. Exemplary preferred xanthine compounds for use as an MrgprX4 antagonist as set forth herein are disclosed in WO2022/079245 (PCT/EP2021/078622), including compounds B-1 to B- [symbol] -D3, E1-E3, F1, G1-G3 and J1-J3 as disclosed in WO [symbol] (PCT/EP2021/078622) which is incorporated by reference herein in its entirety.

Exemplary preferred xanthine compounds that may be suitably employed in the present methods and compostions also include those shown in the following Table 1.

TABLE 1

| Compound | |
|---|---|
| No. | Structure |
| 1 | 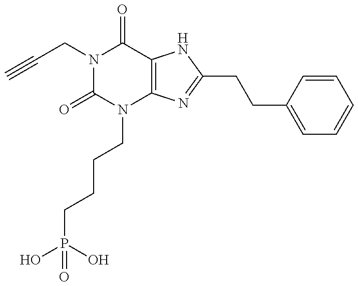 [SMILES: C#CCn1c(=O)c2[nH]c(CCc3ccccc3)nc2n(CCCCP(=O)(O)O)c1=O] |
| 2 | 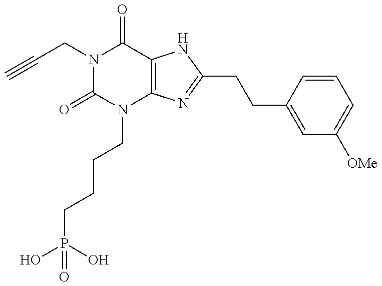 [SMILES: C#CCn1c(=O)c2[nH]c(CCc3cccc(OC)c3)nc2n(CCCCP(=O)(O)O)c1=O] |
| 3 | 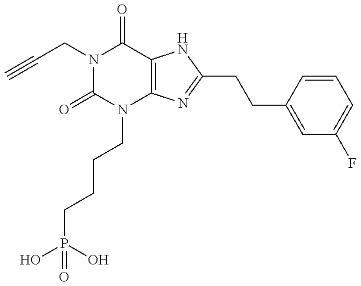 [SMILES: C#CCn1c(=O)c2[nH]c(CCc3cccc(F)c3)nc2n(CCCCP(=O)(O)O)c1=O] |
| 4 | 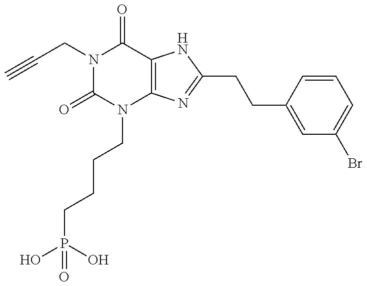 [SMILES: C#CCn1c(=O)c2[nH]c(CCc3cccc(Br)c3)nc2n(CCCCP(=O)(O)O)c1=O] |
| 5 | 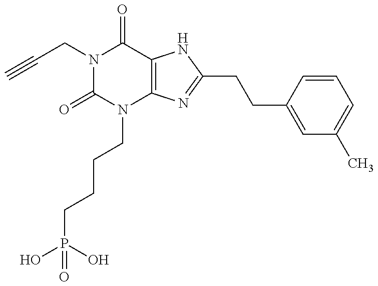 [SMILES: C#CCn1c(=O)c2[nH]c(CCc3cccc(C)c3)nc2n(CCCCP(=O)(O)O)c1=O] |
| 6 | 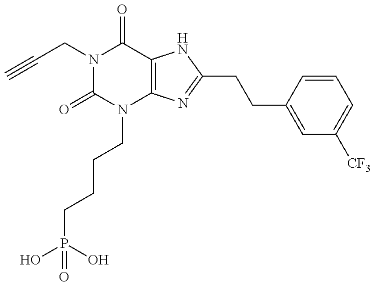 [SMILES: C#CCn1c(=O)c2[nH]c(CCc3cccc(C(F)(F)F)c3)nc2n(CCCCP(=O)(O)O)c1=O] |
| 7 | 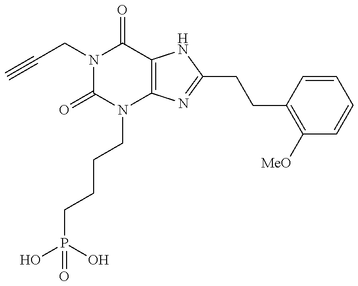 [SMILES: C#CCn1c(=O)c2[nH]c(CCc3ccccc3OC)nc2n(CCCCP(=O)(O)O)c1=O] |
| 8 | 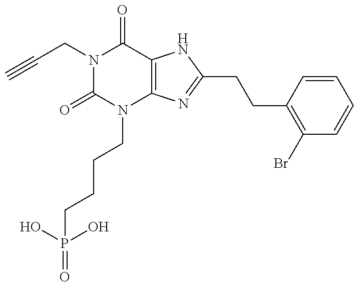 [SMILES: C#CCn1c(=O)c2[nH]c(CCc3ccccc3Br)nc2n(CCCCP(=O)(O)O)c1=O] |
| 0 | |
| 9 | 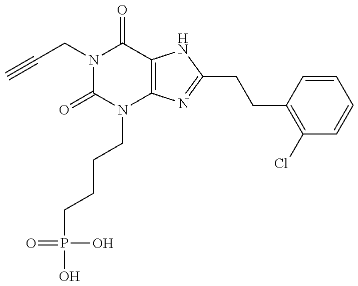 [SMILES: C#CCn1c(=O)c2[nH]c(CCc3ccccc3Cl)nc2n(CCCCP(=O)(O)O)c1=O] |
| 10 | 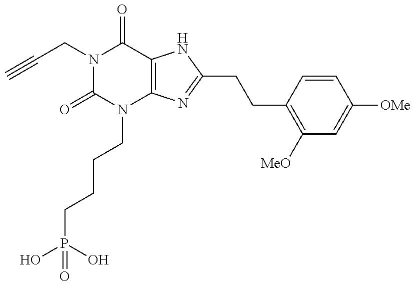 [SMILES: C#CCn1c(=O)c2[nH]c(CCc3ccc(OC)cc3OC)nc2n(CCCCP(=O)(O)O)c1=O] |
| 11 | 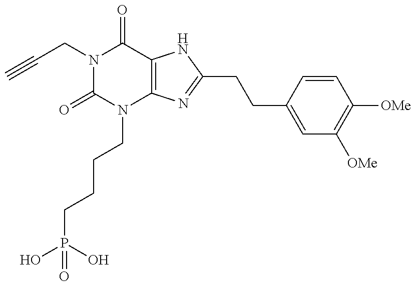 [SMILES: C#CCn1c(=O)c2[nH]c(CCc3ccc(OC)c(OC)c3)nc2n(CCCCP(=O)(O)O)c1=O] |
| 12 | 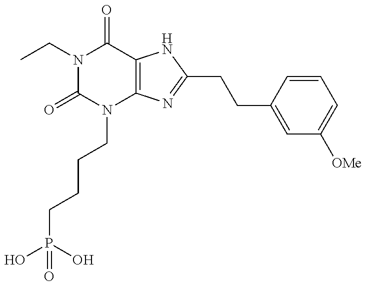 [SMILES: CCn1c(=O)c2[nH]c(CCc3cccc(OC)c3)nc2n(CCCCP(=O)(O)O)c1=O] |
| 13 | 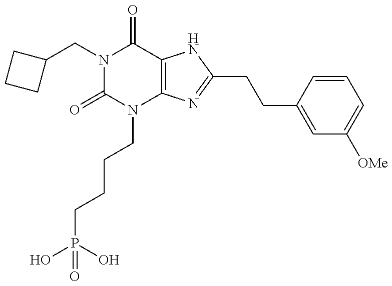 [SMILES: COc1cccc(CCc2nc3c([nH]2)c(=O)n(CC2CCC2)c(=O)n3CCCCP(=O)(O)O)c1] |
| 14 | 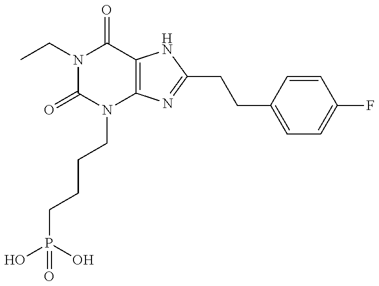 [SMILES: CCn1c(=O)c2[nH]c(CCc3ccc(F)cc3)nc2n(CCCCP(=O)(O)O)c1=O] |
| 15 | 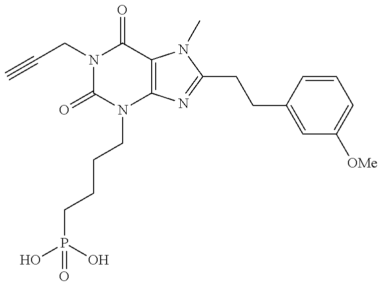 [SMILES: C#CCn1c(=O)c2c(nc(CCc3cccc(OC)c3)n2C)n(CCCCP(=O)(O)O)c1=O] |
| 16 | 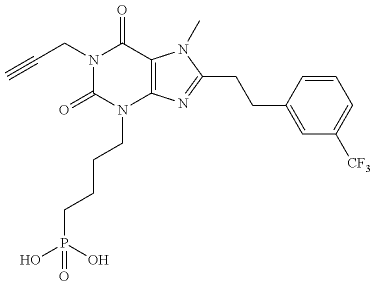 [SMILES: C#CCn1c(=O)c2c(nc(CCc3cccc(C(F)(F)F)c3)n2C)n(CCCCP(=O)(O)O)c1=O] |
| 17 | 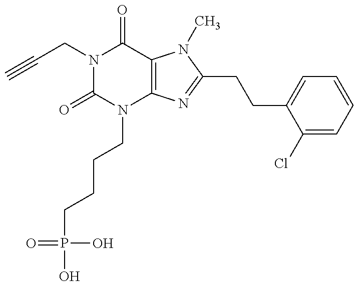 [SMILES: C#CCn1c(=O)c2c(nc(CCc3ccccc3Cl)n2C)n(CCCCP(=O)(O)O)c1=O] |
| 18 | 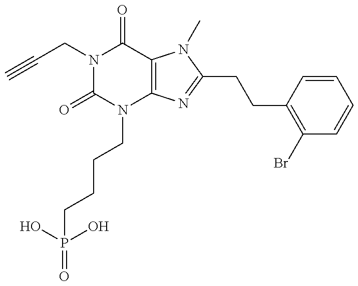 [SMILES: C#CCn1c(=O)c2c(nc(CCc3ccccc3Br)n2C)n(CCCCP(=O)(O)O)c1=O] |
| 19 | 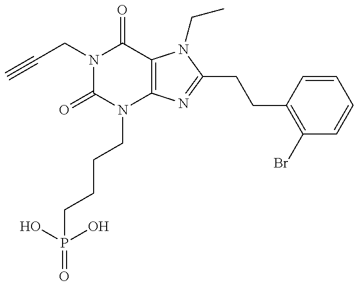 [SMILES: C#CCn1c(=O)c2c(nc(CCc3ccccc3Br)n2CC)n(CCCCP(=O)(O)O)c1=O] |
| 20 | 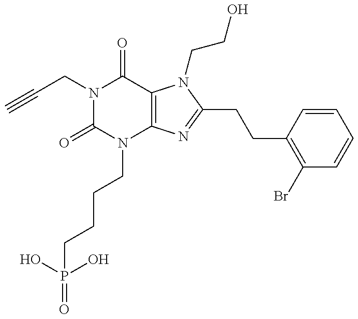 [SMILES: C#CCn1c(=O)c2c(nc(CCc3ccccc3Br)n2CCO)n(CCCCP(=O)(O)O)c1=O] |
| 21 | 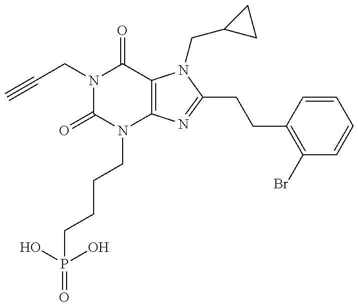 [SMILES: C#CCn1c(=O)c2c(nc(CCc3ccccc3Br)n2CC2CC2)n(CCCCP(=O)(O)O)c1=O] |
| 22 | 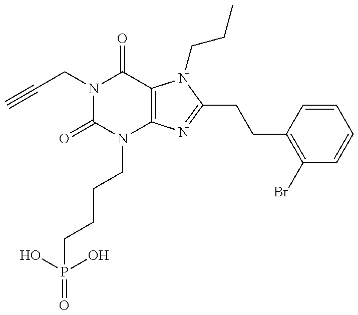 [SMILES: C#CCn1c(=O)c2c(nc(CCc3ccccc3Br)n2CCC)n(CCCCP(=O)(O)O)c1=O] |
| 23 | 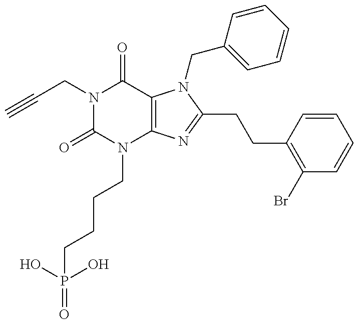 [SMILES: C#CCn1c(=O)c2c(nc(CCc3ccccc3Br)n2Cc2ccccc2)n(CCCCP(=O)(O)O)c1=O] |
| 24 | 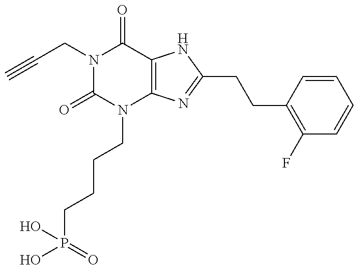 [SMILES: C#CCn1c(=O)c2[nH]c(CCc3ccccc3F)nc2n(CCCCP(=O)(O)O)c1=O] |
| 25 | 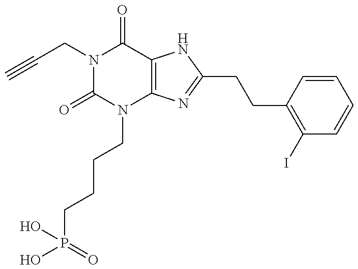 [SMILES: C#CCn1c(=O)c2[nH]c(CCc3ccccc3I)nc2n(CCCCP(=O)(O)O)c1=O] |
| 26 | 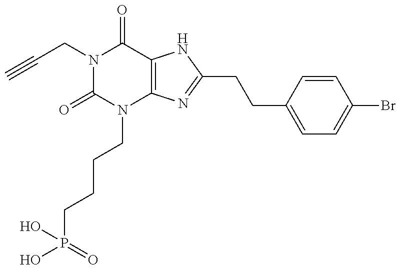 [SMILES: C#CCn1c(=O)c2[nH]c(CCc3ccc(Br)cc3)nc2n(CCCCP(=O)(O)O)c1=O] |
| 27 | 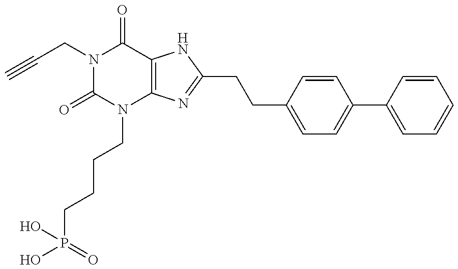 [SMILES: C#CCn1c(=O)c2[nH]c(CCc3ccc(-c4ccccc4)cc3)nc2n(CCCCP(=O)(O)O)c1=O] |
| 28 | 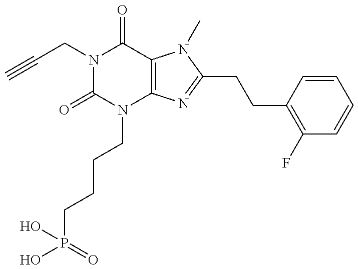 [SMILES: C#CCn1c(=O)c2c(nc(CCc3ccccc3F)n2C)n(CCCCP(=O)(O)O)c1=O] |
| 29 | 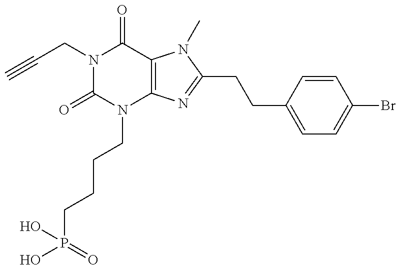 [SMILES: C#CCn1c(=O)c2c(nc(CCc3ccc(Br)cc3)n2C)n(CCCCP(=O)(O)O)c1=O] |
| 30 | 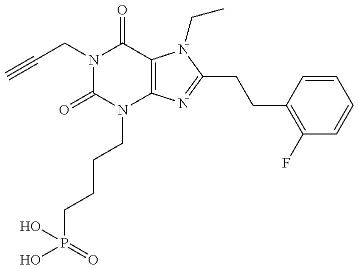 [SMILES: C#CCn1c(=O)c2c(nc(CCc3ccccc3F)n2CC)n(CCCCP(=O)(O)O)c1=O] |
| 31 | 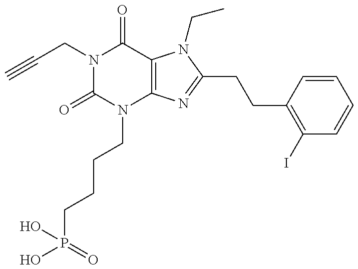 [SMILES: C#CCn1c(=O)c2c(nc(CCc3ccccc3I)n2CC)n(CCCCP(=O)(O)O)c1=O] |
| 32 | 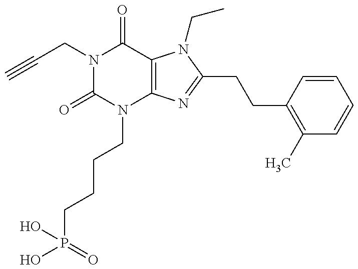 [SMILES: C#CCn1c(=O)c2c(nc(CCc3ccccc3C)n2CC)n(CCCCP(=O)(O)O)c1=O] |
| 33 | 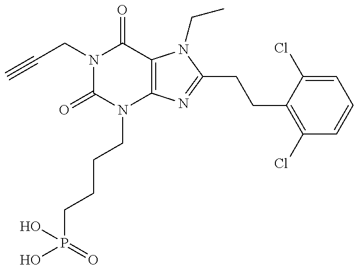 [SMILES: C#CCn1c(=O)c2c(nc(CCc3c(Cl)cccc3Cl)n2CC)n(CCCCP(=O)(O)O)c1=O] |
| 34 | 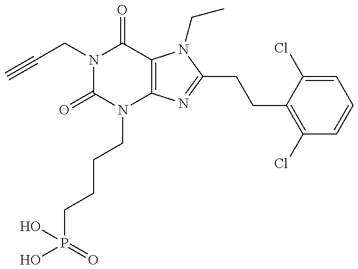 [SMILES: C#CCn1c(=O)c2c(nc(CCc3c(Cl)cccc3Cl)n2CC)n(CCCCP(=O)(O)O)c1=O] |
| 35 | 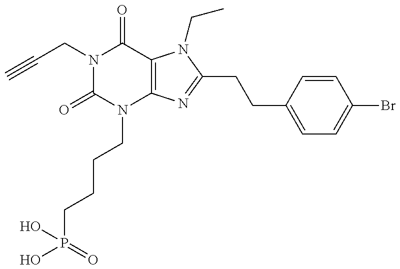 [SMILES: C#CCn1c(=O)c2c(nc(CCc3ccc(Br)cc3)n2CC)n(CCCCP(=O)(O)O)c1=O] |
| 36 | 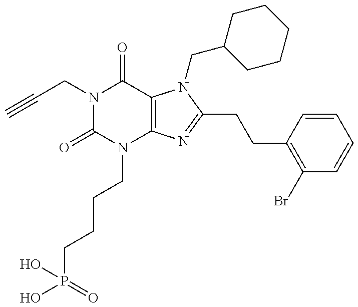 [SMILES: C#CCn1c(=O)c2c(nc(CCc3ccccc3Br)n2CC2CCCCC2)n(CCCCP(=O)(O)O)c1=O] |
| 37 | 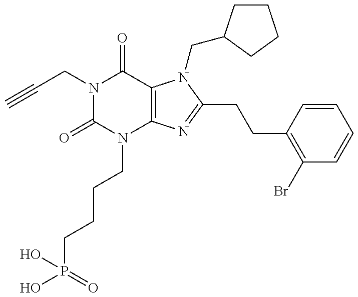 [SMILES: C#CCn1c(=O)c2c(nc(CCc3ccccc3Br)n2CC2CCCC2)n(CCCCP(=O)(O)O)c1=O] |
| 38 | 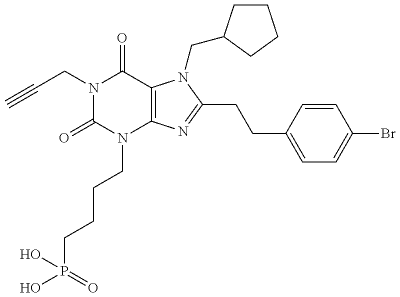 [SMILES: C#CCn1c(=O)c2c(nc(CCc3ccc(Br)cc3)n2CC2CCCC2)n(CCCCP(=O)(O)O)c1=O] |
| 39 | 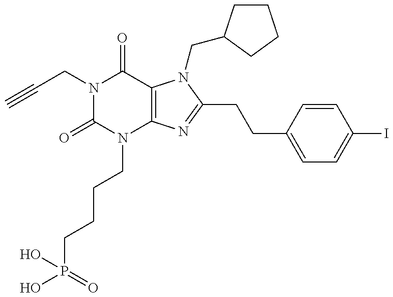 [SMILES: C#CCn1c(=O)c2c(nc(CCc3ccc(I)cc3)n2CC2CCCC2)n(CCCCP(=O)(O)O)c1=O] |
| 40 | 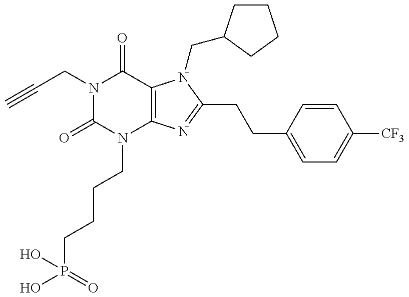 [SMILES: C#CCn1c(=O)c2c(nc(CCc3ccc(C(F)(F)F)cc3)n2CC2CCCC2)n(CCCCP(=O)(O)O)c1=O] |
| 41 | 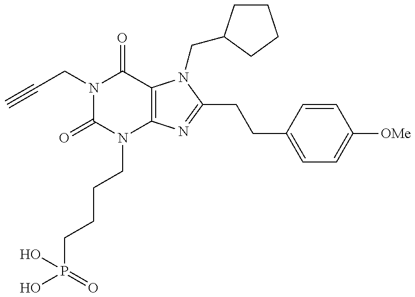 [SMILES: C#CCn1c(=O)c2c(nc(CCc3ccc(OC)cc3)n2CC2CCCC2)n(CCCCP(=O)(O)O)c1=O] |
| 42 | 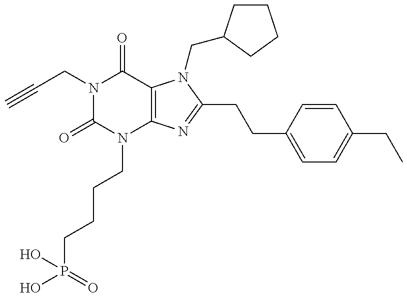 [SMILES: C#CCn1c(=O)c2c(nc(CCc3ccc(CC)cc3)n2CC2CCCC2)n(CCCCP(=O)(O)O)c1=O] |
| 43 | 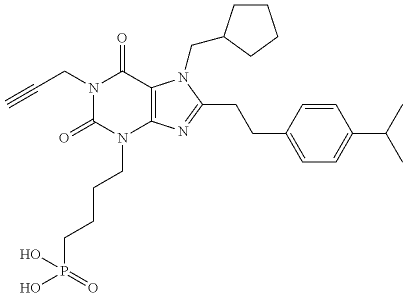 [SMILES: C#CCn1c(=O)c2c(nc(CCc3ccc(C(C)C)cc3)n2CC2CCCC2)n(CCCCP(=O)(O)O)c1=O] |
| 44 | 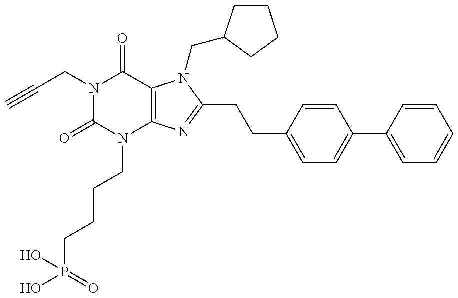 [SMILES: C#CCn1c(=O)c2c(nc(CCc3ccc(-c4ccccc4)cc3)n2CC2CCCC2)n(CCCCP(=O)(O)O)c1=O] |
| 45 | 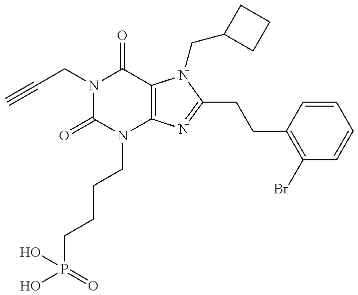 [SMILES: C#CCn1c(=O)c2c(nc(CCc3ccccc3Br)n2CC2CCC2)n(CCCCP(=O)(O)O)c1=O] |
| 46 | 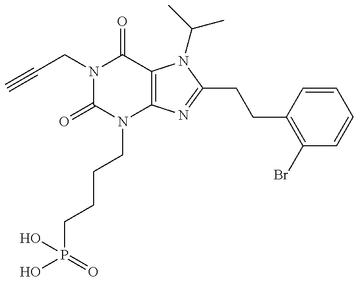 [SMILES: C#CCn1c(=O)c2c(nc(CCc3ccccc3Br)n2C(C)C)n(CCCCP(=O)(O)O)c1=O] |
| 47 | 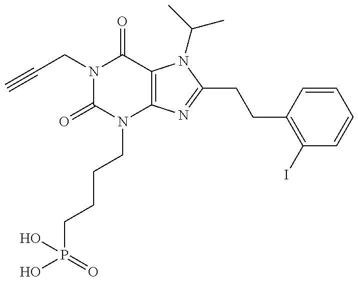 [SMILES: C#CCn1c(=O)c2c(nc(CCc3ccccc3I)n2C(C)C)n(CCCCP(=O)(O)O)c1=O] |
| 48 | 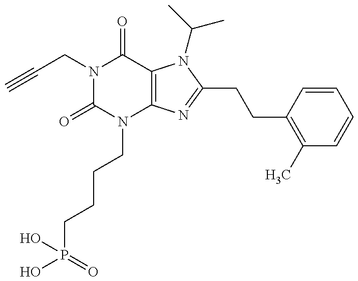 [SMILES: C#CCn1c(=O)c2c(nc(CCc3ccccc3C)n2C(C)C)n(CCCCP(=O)(O)O)c1=O] |
| 49 | 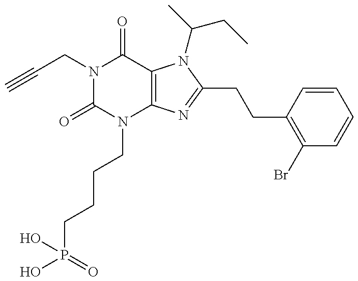 [SMILES: C#CCn1c(=O)c2c(nc(CCc3ccccc3Br)n2C(C)CC)n(CCCCP(=O)(O)O)c1=O] |
| 50 | 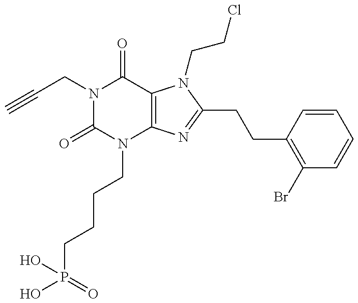 [SMILES: C#CCn1c(=O)c2c(nc(CCc3ccccc3Br)n2CCCl)n(CCCCP(=O)(O)O)c1=O] |
| 51 | 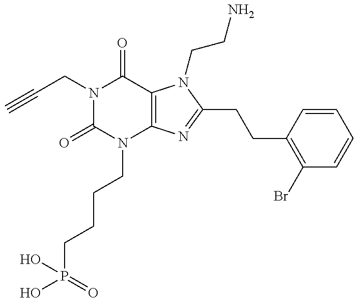 [SMILES: C#CCn1c(=O)c2c(nc(CCc3ccccc3Br)n2CCN)n(CCCCP(=O)(O)O)c1=O] |
| 52 | 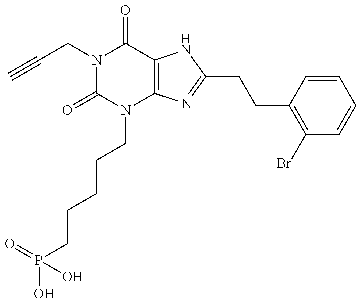 [SMILES: C#CCn1c(=O)c2[nH]c(CCc3ccccc3Br)nc2n(CCCCCP(=O)(O)O)c1=O] |
| 53 | 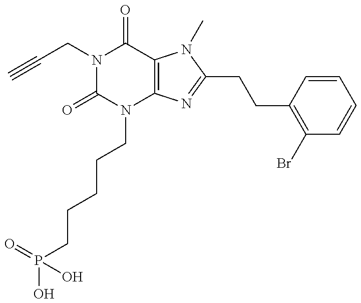 [SMILES: C#CCn1c(=O)c2c(nc(CCc3ccccc3Br)n2C)n(CCCCCP(=O)(O)O)c1=O] |
| 54 | 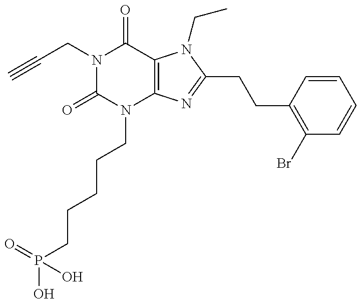 [SMILES: C#CCn1c(=O)c2c(nc(CCc3ccccc3Br)n2CC)n(CCCCCP(=O)(O)O)c1=O] |
| 55 | 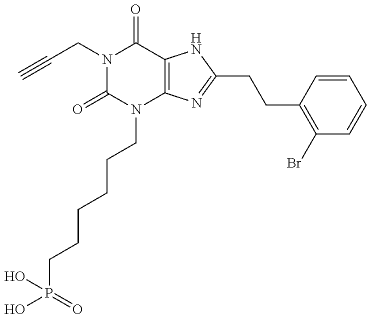 [SMILES: C#CCn1c(=O)c2[nH]c(CCc3ccccc3Br)nc2n(CCCCCCP(=O)(O)O)c1=O] |
| 56 | 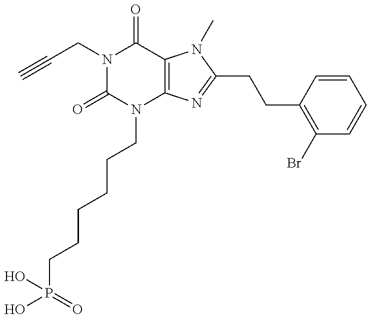 [SMILES: C#CCn1c(=O)c2c(nc(CCc3ccccc3Br)n2C)n(CCCCCCP(=O)(O)O)c1=O] |
| 57 | 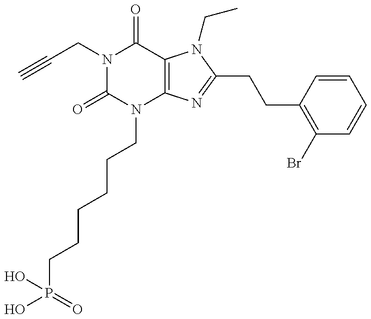 [SMILES: C#CCn1c(=O)c2c(nc(CCc3ccccc3Br)n2CC)n(CCCCCCP(=O)(O)O)c1=O] |
| 58 | 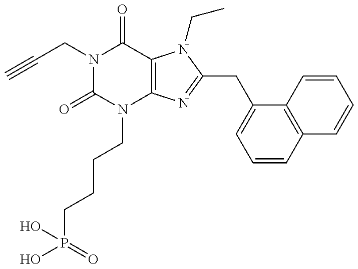 [SMILES: C#CCn1c(=O)c2c(nc(Cc3cccc4ccccc34)n2CC)n(CCCCP(=O)(O)O)c1=O] |
| 59 | 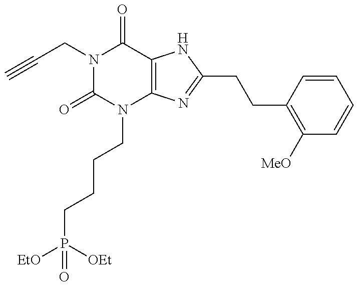 [SMILES: C#CCn1c(=O)c2[nH]c(CCc3ccccc3OC)nc2n(CCCCP(=O)(OCC)OCC)c1=O] |
| 60 | 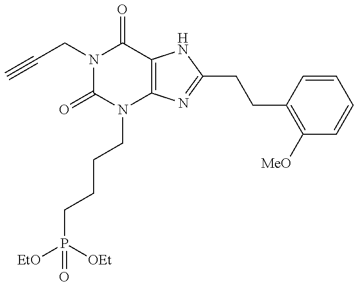 [SMILES: C#CCn1c(=O)c2[nH]c(CCc3ccccc3OC)nc2n(CCCCP(=O)(OCC)OCC)c1=O] |
| 61 | 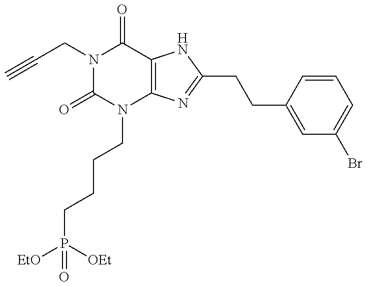 [SMILES: C#CCn1c(=O)c2[nH]c(CCc3cccc(Br)c3)nc2n(CCCCP(=O)(OCC)OCC)c1=O] |
| 62 | 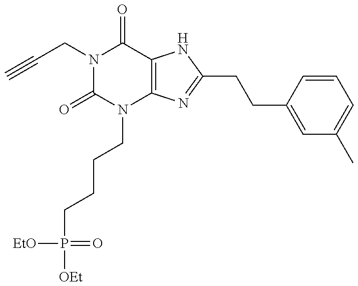 [SMILES: C#CCn1c(=O)c2[nH]c(CCc3cccc(C)c3)nc2n(CCCCP(=O)(OCC)OCC)c1=O] |
| 63 | 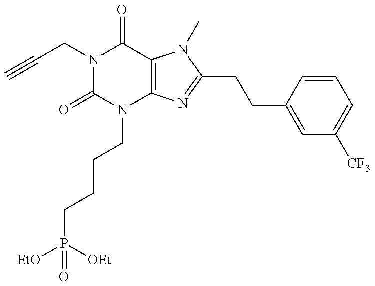 [SMILES: C#CCn1c(=O)c2c(nc(CCc3cccc(C(F)(F)F)c3)n2C)n(CCCCP(=O)(OCC)OCC)c1=O] |
| 64 | 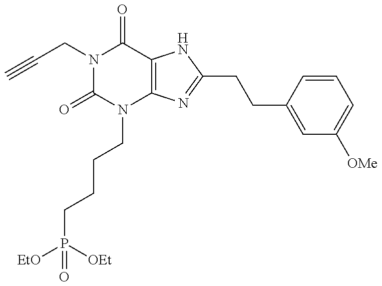 [SMILES: C#CCn1c(=O)c2[nH]c(CCc3cccc(OC)c3)nc2n(CCCCP(=O)(OCC)OCC)c1=O] |
| 65 | 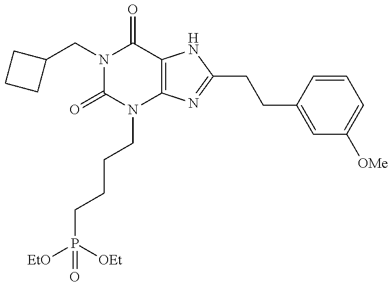 [SMILES: CCOP(=O)(CCCCn1c(=O)n(CC2CCC2)c(=O)c2[nH]c(CCc3cccc(OC)c3)nc21)OCC] |
| 66 | 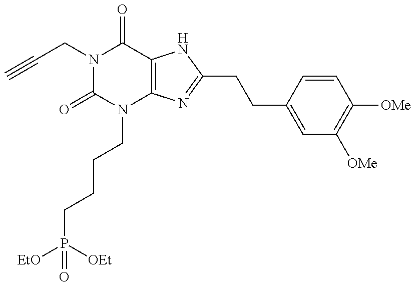 [SMILES: C#CCn1c(=O)c2[nH]c(CCc3ccc(OC)c(OC)c3)nc2n(CCCCP(=O)(OCC)OCC)c1=O] |
| 67 | 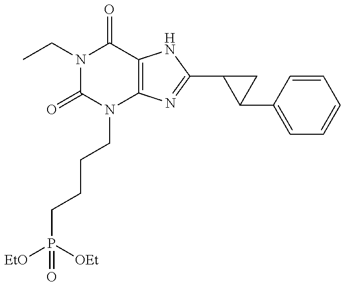 [SMILES: CCOP(=O)(CCCCn1c(=O)n(CC)c(=O)c2[nH]c(C3CC3c3ccccc3)nc21)OCC] |
| 68 | 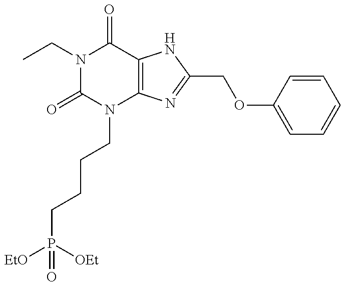 [SMILES: CCOP(=O)(CCCCn1c(=O)n(CC)c(=O)c2[nH]c(COc3ccccc3)nc21)OCC] |
| 69 | 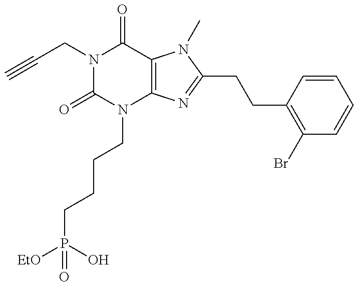 [SMILES: C#CCn1c(=O)c2c(nc(CCc3ccccc3Br)n2C)n(CCCCP(=O)(O)OCC)c1=O] |
| 70 | 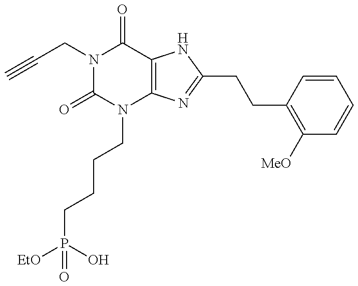 [SMILES: C#CCn1c(=O)c2[nH]c(CCc3ccccc3OC)nc2n(CCCCP(=O)(O)OCC)c1=O] |
| 71 | 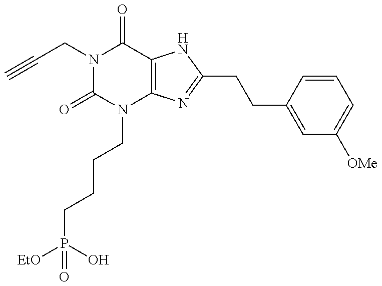 [SMILES: C#CCn1c(=O)c2[nH]c(CCc3cccc(OC)c3)nc2n(CCCCP(=O)(O)OCC)c1=O] |
| 72 | 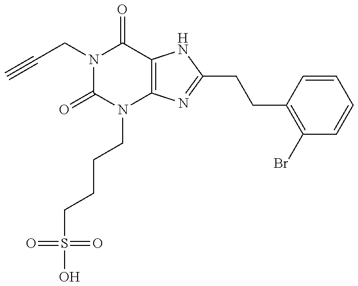 [SMILES: C#CCn1c(=O)c2[nH]c(CCc3ccccc3Br)nc2n(CCCCS(=O)(=O)O)c1=O] |
| 73 | 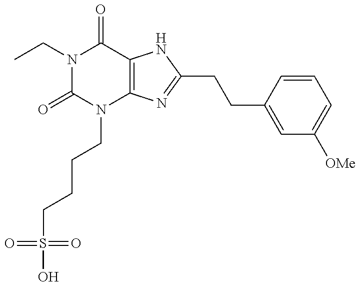 [SMILES: CCn1c(=O)c2[nH]c(CCc3cccc(OC)c3)nc2n(CCCCS(=O)(=O)O)c1=O] |
| 74 | 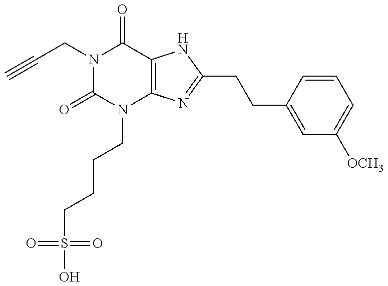 [SMILES: C#CCn1c(=O)c2[nH]c(CCc3cccc(OC)c3)nc2n(CCCCS(=O)(=O)O)c1=O] |
| 75 | 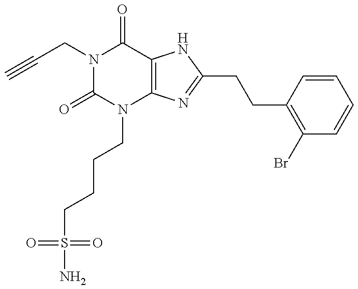 [SMILES: C#CCn1c(=O)c2[nH]c(CCc3ccccc3Br)nc2n(CCCCS(N)(=O)=O)c1=O] |
| 76 | 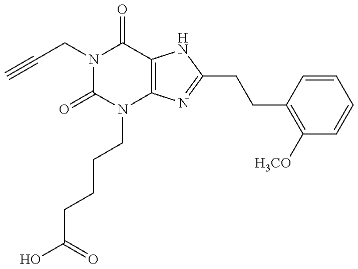 [SMILES: C#CCn1c(=O)c2[nH]c(CCc3ccccc3OC)nc2n(CCCCC(=O)O)c1=O] |
| 77 | 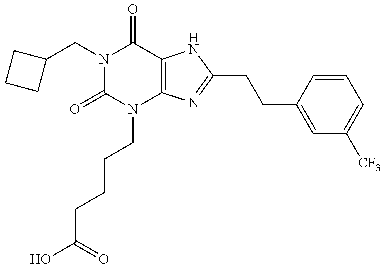 [SMILES: O=C(O)CCCCn1c(=O)n(CC2CCC2)c(=O)c2[nH]c(CCc3cccc(C(F)(F)F)c3)nc21] |
| 78 | 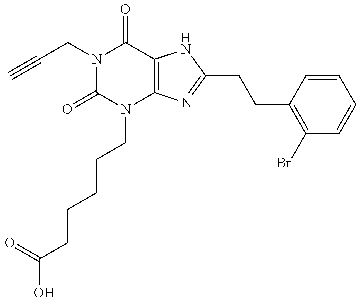 [SMILES: C#CCn1c(=O)c2[nH]c(CCc3ccccc3Br)nc2n(CCCCCC(=O)O)c1=O] |
| 79 | 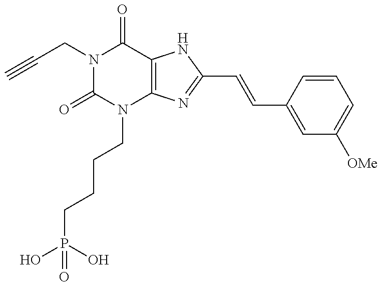 [SMILES: C#CCn1c(=O)c2[nH]c(/C=C/c3cccc(OC)c3)nc2n(CCCCP(=O)(O)O)c1=O] |
| 80 | 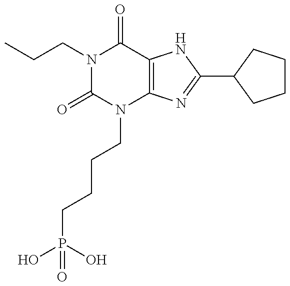 [SMILES: CCCn1c(=O)c2[nH]c(C3CCCC3)nc2n(CCCCP(=O)(O)O)c1=O] |
| 81 | 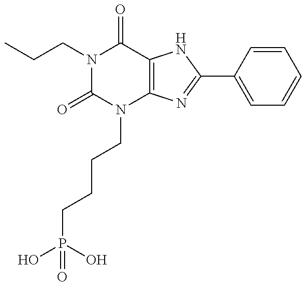 [SMILES: CCCn1c(=O)c2[nH]c(-c3ccccc3)nc2n(CCCCP(=O)(O)O)c1=O] |

The above compounds 1 through 81 in Table 1 can be prepared as disclosed WO2022/079245 (PCT/EP2021/078622).

Additional MrgprX4 antagonist for use in the present methods and compositions including MrgprX4 antagonist small molecules for use in the present methods and compositions are disclosed in WO2020/198537 (PCT/US2020/025077), incorporated herein by reference in its entirety.

Additional MrgprX4 antagonist for use in the present methods and compositions including MrgprX4 antagonist small molecules for use in the present methods and compositions are disclosed in US2020/0173985, incorporated herein by reference in its entirety.

Any of the above compounds also can be further evaluated for efficiency in a particular treatment method or composition by an assay as disclosed herein, include an assay of Example 9 of WO2018/232316, where activation can be assessed by calcium imaging or inositol phosphate or β-arrestin recruitment based detection.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl par [symbol] ants such as ascorbic acid or sodium bisulfite; chelatin [symbol] ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the pu [symbol] rapeutic administration, the active compound can be i [symbol] excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The compositions of the invention could also be formulated as nanoparticle formulations. The compounds of the invention can be administered for immediate-release, delayed-release, modified-release, sustained-release, pulsed-release and/or controlled-release applications. The pharmaceutical compositions of the invention may contain from 0.01 to 99% weight-per volume of the active material. For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including lip [symbol] to infected cells with monoclonal antibodies to viral a [symbol] e used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For a compound used in a method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an MrgprX4 antagonist (i.e., an effective dosage) depends on the agent selected. For instance, single dose amounts of an MrgprX4 antagonist in the range of approximately 1 pg to 1000 mg per day may be administered; in some embodiments, 10, 30, 100, or 1000 pg, or 10, 30, 100, or 1000 ng, or 10, 30, 100, or 1000 μg, or 10, 30, 100, or 1000 mg per day may be administered.

A therapeutically effective amount of the compound of the present invention can be determined by methods known in the art. In addition to depending on the agent and selected/pharmaceutical formulation used, the therapeutically effective quantities of a pharmaceutical composition of the invention will depend on the age and on the general physiological condition of the patient and the route of administration. In certain embodiments, the therapeutic doses will generally be between about 10 and 2000 mg/day and preferably between about 30 and 1500 mg/day. Other ranges may be used, including, for ex [symbol] g/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an agent can include a single treatment or, optionally, can include a series of treatments.

It can be appreciated that the method of introducing an agent into the environment of a cell will depend on the type of cell and the makeup of its environment. Suitable amounts of an agent must be introduced and these amounts can be empirically determined using standard methods. Exemplary effective concentrations of an individual agent in the environment of a cell can be 500 millimolar or less, 50 millimolar or less, 10 millimolar or less, 1 millimolar or less, 500 nanomolar or less, 50 nanomolar or less, 10 nanomolar or less, or even compositions in which concentrations of 1 nanomolar or less can be used.

The pharmaceutical compositions can be included in a kit, container, pack, or dispenser together with instructions for administration.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

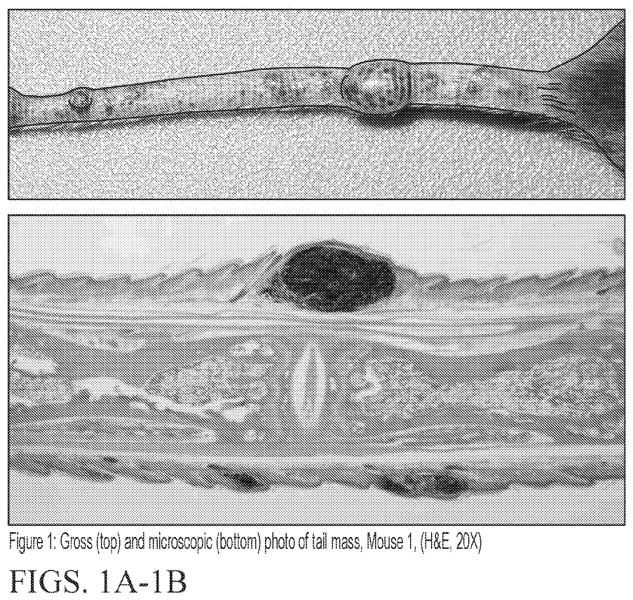

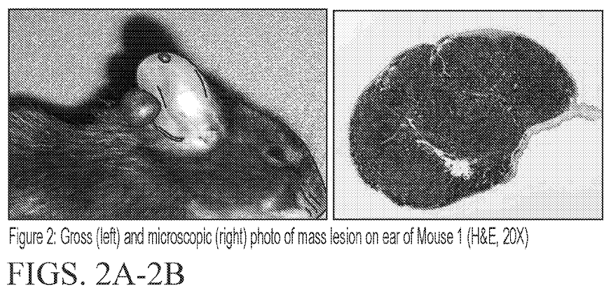

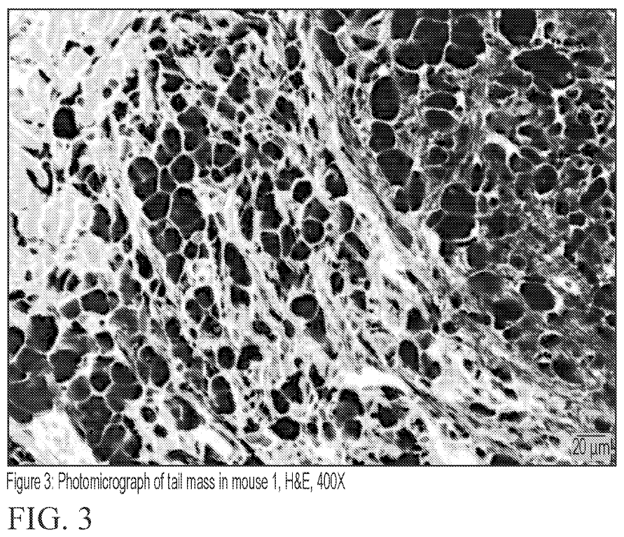

Mice overexpressing MRGPRX4 under S100A8Cre driver were examined by histopathological analysis to access the skin lesions. The histopathologist confirmed the growths (approx. 0.1-0.5 cm) on mice tails and ear pinnae as melanoma. FIGS. 1-3 depict gross and microscopic images of tumors from the test subjects tails and pinnae.

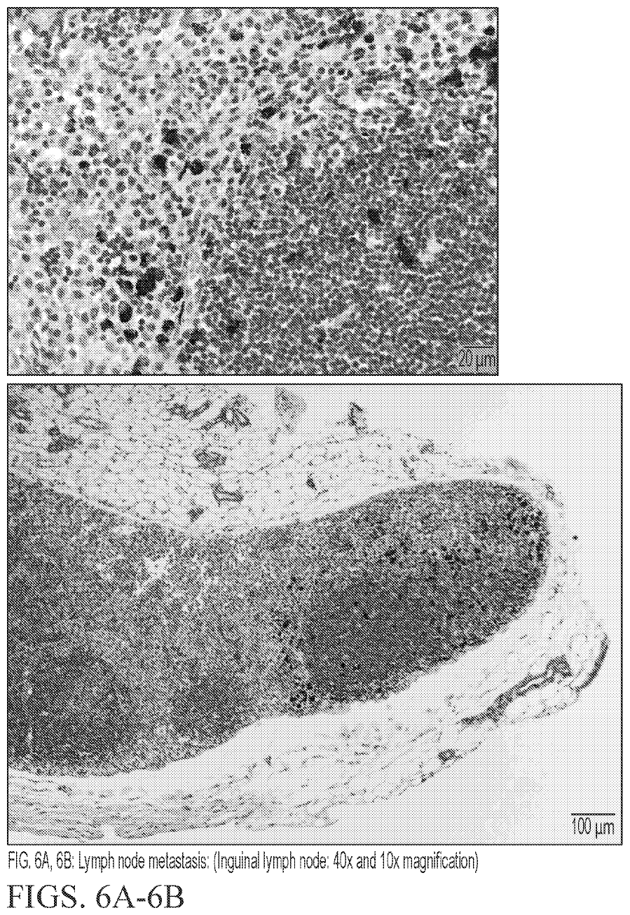

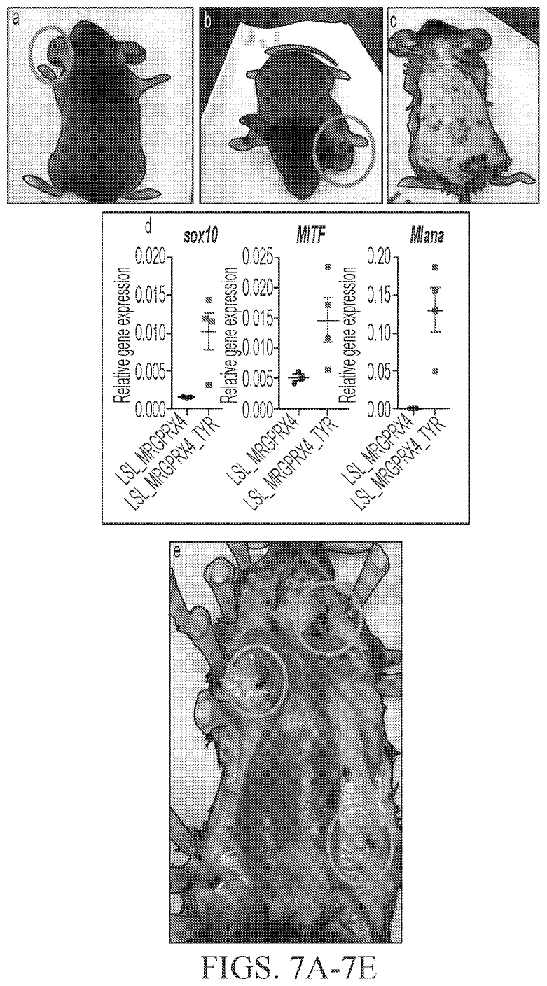

Gross necropsy report highlights that bilateral inguinal lymph nodes in these mice shows a discolored, focal, black morphology (as show in FIGS. 6A, 6B). Thus, these results indicate overexpression of MRGPRX4 leads to not only the development of cutaneous me [symbol] also promote the egress of melanoma cells into the ly [symbol] suggesting the possibility of metastasis.

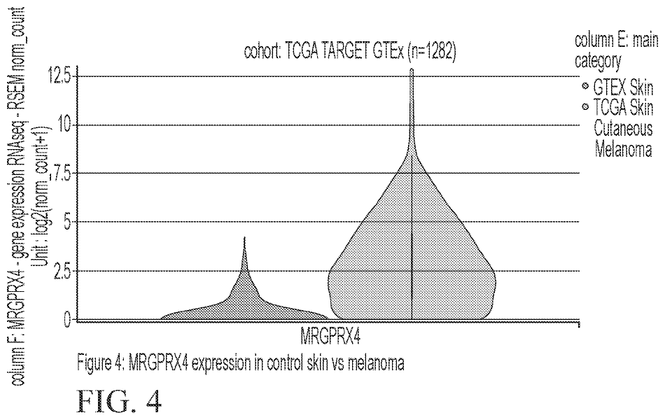

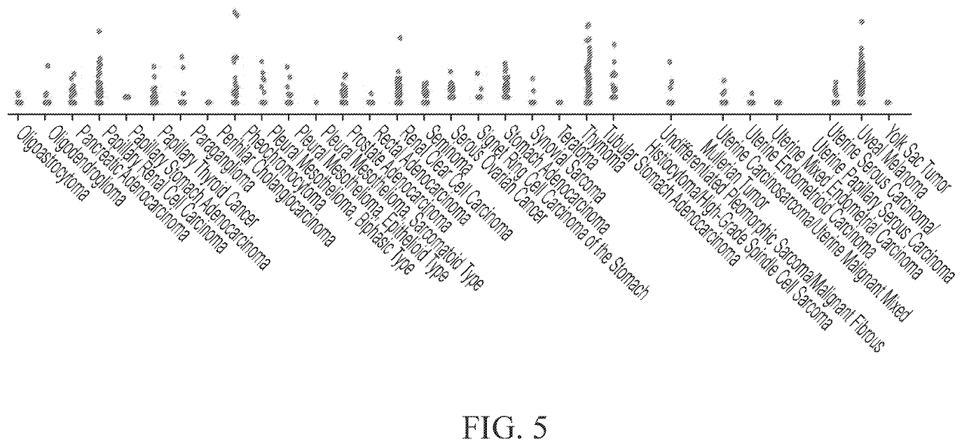

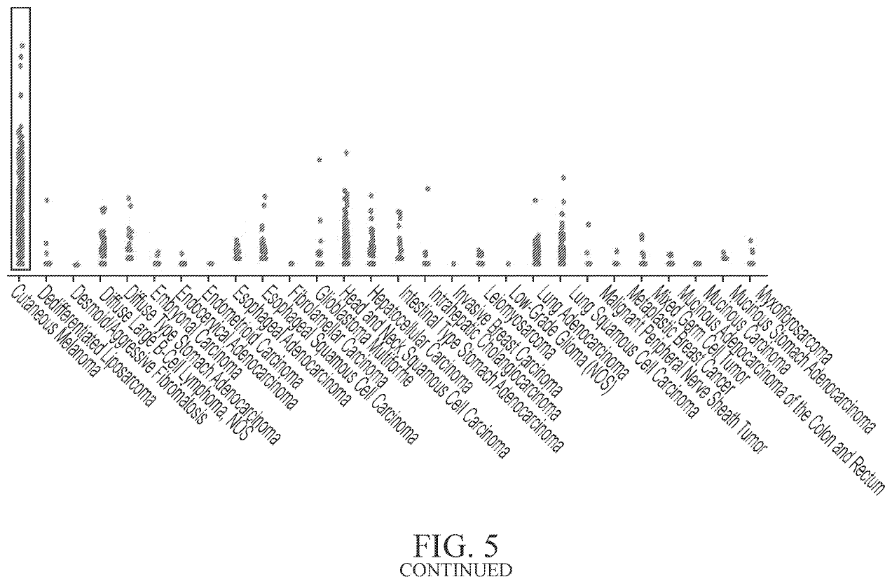

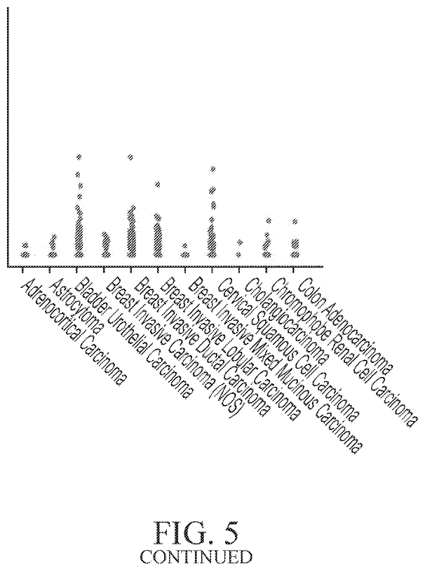

Based on our findings in mice where overexpression of the single gene, MRGPRX4, leads to spontaneous pathology we hypothesized that MRGPRX4 may be an oncogene and its dysregulated expression may be a driver of melanoma. To this end, we performed data mining of The Cancer Genome Atlas (TCGA) to assess levels of MRGPRX4 in human melanoma. It was found that as compared to control skin, melanoma samples express very high levels of MRGPRX4 (p<10⁻⁸⁴) as shown in FIG. 4. Moreover, specifically when we assess levels of MRGPRX4 in various cancers (using TCGA dataset), we see highest expression in melanoma, see FIG. 5 (cutaneous melanoma highlighted in red).

### Example 2: MRGPRX4 Expression in Melanocyte Results in Melanoma

To specifically target MRGPRX4 in all melanocytes, we bred our Rosa26LSL-MRGPRX4 mice with B6.Cg-Tg (Tyr-cre/ERT2) 13Bos/J (Stock #012328) from The Jackson Laboratory. The tyrosinase-cre driver is the most robust method to drive the expression of the gene of interest in all melanocytes. The resulting progeny were treated with tamoxifen (TAM: 1 mg/40 g, 3 consecutives, 100 ul/corn oil i.p. injections) to induce Cre activation. Following this, mice were monitored for tumor growth. Only the mice overexpressing MRGPRX4 in melanocytes (LSL-MRGPRX4_TYR) and not the control mice (LSL-MRGPRX4), developed palpable tumors, that can be seen approximately two weeks after TAM injection. For analysis of metastasis, mice were euthanized, and lymph nodes and organs were examined. Mice overexpressing MRGPRX4 in melanocytes (LSL-MRGPRX4_TYR) develop robust malignant skin melanoma phenotype with 100% penetrance.

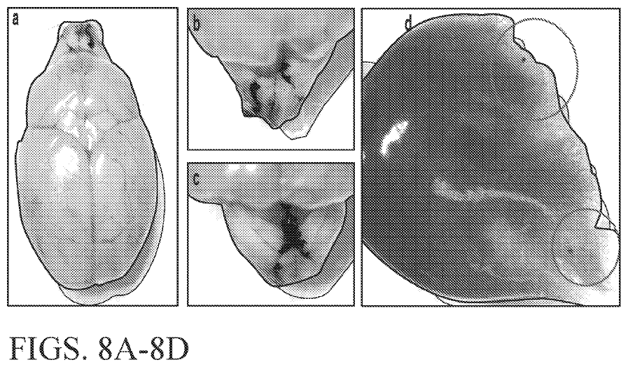

Results are further shown in FIGS. 7 and 8. In FIG. 7, the depicted images are from mice approximately 45 days post TAM injection. Mice develop tumors on skin of ear, tail, back (see FIGS. 7a-c). Melanoma skin lesions from these mice depict high expression of melanoma genes such as sox10, MITF and Mlana, compared to control skin (FIG. 7d). Dissection of the mice depict that lymph nodes (cervical, axial, brachial, inguinal) harbor melanoma cells as shown in FIG. 7e.

In FIG. 8, mice overexpressing MRGPRX4 in melanocytes (LSL_MRGPRX4_Tyr) also show metastatic cells in distal organs such as the brain (see FIGS. 8a-c) and lungs (see FIG. 8d).

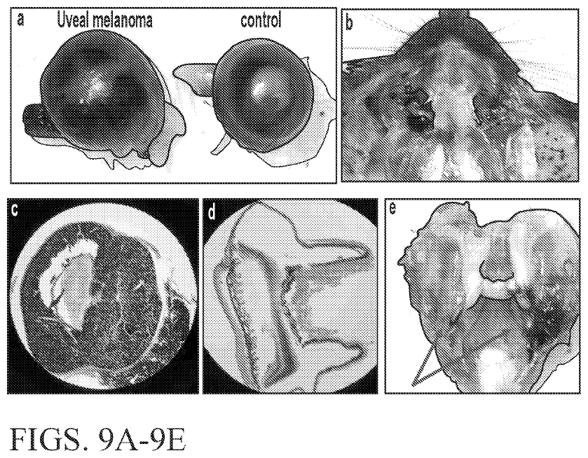

### Example 3: Mice Overexpressing MRGPRX4 in Melanocytes Develop Uveal and Meningeal Melanoma

[symbol] were overexpressing MRGPRX4 in melanocytes (LSL_ [symbol] were seen to develop uveal melanoma as depicted in FIG. 9. FIG. 9a is a gross image of eye with tumor compared to control, FIG. 9b is a tumor behind eye after eye removal, FIG. 9c is H & E of eye melanoma as compared to FIG. 9d which is a control eye. Mice overexpressing MRGPRX4 also develop meningeal melanoma as depicted in FIG. 9e: melanoma in the mouse meninges (refer to arrows).

### Example 4

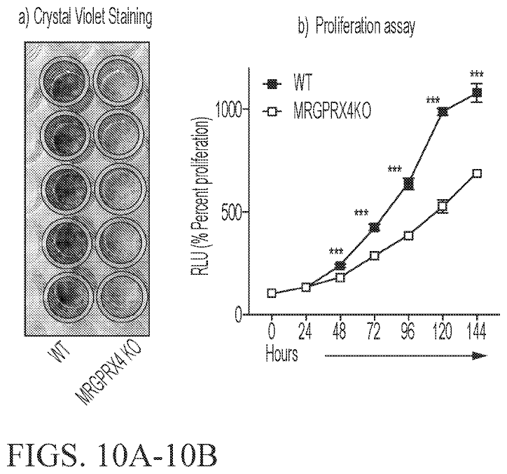

To test the role of MRGPRX4 in human melanoma pathogenesis, a human melanoma cell line deficient in MRGPRX4 signaling (MRGPRX4 knockout (KO)) was generated using CRISPR/Cas9 technology. Loss of MRGPRX4 signaling in human melanoma cell line results in decreased cellular proliferation as demonstrated by two different assays: crystal violet stain and ATP measurement. Thus, MRGPRX4 causes melanoma cells to proliferate. Results are shown in FIG. 10.

A human melanoma cell line (A2058) sufficient (WT) or deficient for MRGPRX4 (KO) were seeded (1×10³) in culture media in a 96 well plate. After 5 days, cells were fixed and stained with crystal violet (stains adherent cells in the culture dish, FIG. 10a). For ATP based cellular proliferation assay, cells (2.5×10²) were seeded in a 96 well black plates and ATP levels were measured (Promega Cell Titre Glo) on Day 0 and every day for 6 days. Percent proliferation is calculated based on relative values from day 0 (FIG. 10b).

### Example 5

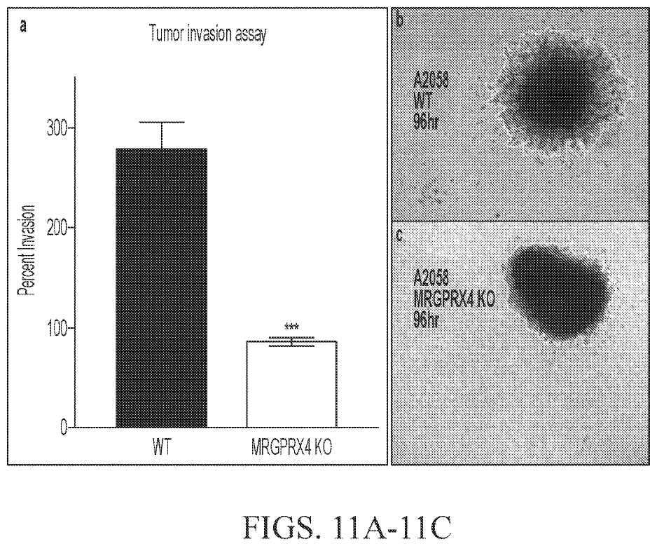

It was found that loss of MRGPRX4 signaling results in decreased melanoma cancer cell line invasion potential. A human melanoma cell line (A2058) sufficient (WT, see FIG. 11a-11b) or deficient for MRGPRX4 (KO, see FIGS. 11a and 11c) were used to form spheroids for 3 days. The spheroids were then seeded in 24 well plates in collagen matrix and cultured in tissue-culture incubator for 96 hours. Images were taken at 2 different time points (0 hr and 96 hr). Percent invasion was assessed using image J and calculated as ((area t₉₆−area tₒ/areatₒ)*100).

### Example 6

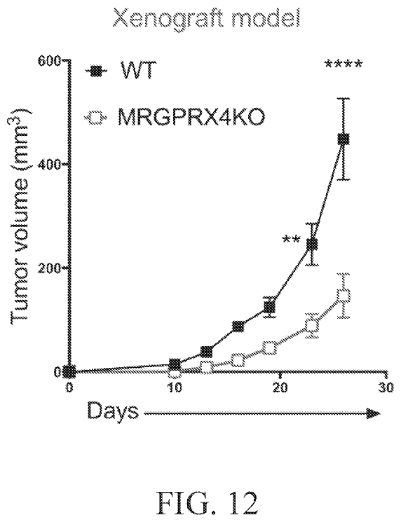

It was found that loss of MRGPRX4 signaling results in decreased melanoma tumor burden in xenograft model in mice in vivo. Thus, a human melanoma cell line (A2058) sufficient (WT, n=7) or deficient MRGPRX4 (KO, n=5) were injected (0.5×10⁶) subcutaneously into immune-deficient Nod Scid Gamma (NSG) mice. Mice were monitored an [symbol] tumors were noticed, measurements were performed u [symbol] or volume was calculated as length*width*width/2. Results are shown in FIG. 12.

### Example 7

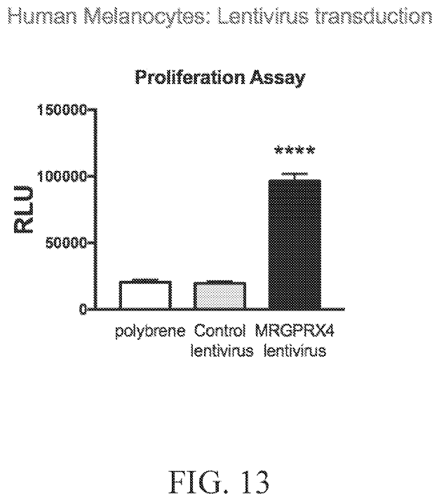

It was found that overexpression of melanocytes using a lentivirus coding for MRGPRX4 causes robust proliferation of human primary melanocytes. Human primary melanocytes were seeded (3×10⁴) in a 24 well tissue-culture plate. Following day (Day 0) cells were transduced (MOI 30) with either control lentivirus or MRGPRX4-lentivirus using polybrene. Following day, media was refreshed and cells were cultured for 6 days for measurement of cellular proliferation using ATP assay (Promega Cell titre Glo). Results are shown in FIG. 13.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating melanoma in a subject, the method comprising:
administering an effective amount of an MrgprX4 antagonist to the subject, thereby treating the melanoma.

2. The method of claim 1 wherein the antagonist comprises an antibody or fragment thereof, a binding protein, a polypeptide, or any combination thereof.

3. The method of claim 1 wherein the antagonist comprises a small molecule.

4. The method of claim 1 wherein the antagonist comprises an antibody or antibody fragment.

5. The method of claim 1 wherein the antagonist is a peptide.

6. The method of claim 1 wherein the antagonist is selected from the group comprising an antibody or fragment thereof, a binding protein, a polypeptide, a small molecule, a nucleic acid, or any combination thereof.

7. The method of claim 1 wherein the subject is identified as suffering from melanoma and the MrgprX4 antagonist is administered to the identified subject.

8. The method of claim 1 wherein the subject is identified as suffering from melanoma of skin and the MrgprX4 antagonist is administered to the identified subject.

9. The method of claim 1 wherein the subject is identified as suffering from melanoma of eyes and the MrgprX4 antagonist is administered to the identified subject.

10. The method of claim 1 wherein the subject is identified as suffering from melanoma of meninges and the MrgprX4 antagonist is administered to the identified subject.

11. A method for diagnosing the presence of melanoma in a subject, comprising:
determining the levels of expression of MrgprX4 in a sample of the subject; and
correlating the levels of MrgprX4 expression in the sample with the presence of melanoma.

12. The method of claim 11 wherein the sample is a tissue sample or a blood sample.

13. A method for screening for drug agents that modulate one or more MrgprX4 associated melanoma comprising:
contacting one or more cells expressing an MrgprX4 G protein coupled receptor with a candidate drug agent; and
detecting a response of the one or more cells to thereby select the candidate drug agent for treatment of melanoma.

14. The method of claim 13 wherein a response of the cells is detected as activation of the G protein coupled receptor.

15. The method of claim 13 wherein the one or more cells are skin cells.

16. The method of claim 13 wherein the one or more cells comprise melanocytes.

17. The method of claim 13 wherein the detected response is an increase in intracellular calcium or activation also can be assessed by inositol phosphate detection or β-arrestin recruitment.

18. A pharmaceutical composition for the treatment of melanoma, the composition comprising an effective amount of an MrgprX4 antagonist.

19-21. (canceled)

22. The pharmaceutical composition of claim 18 wherein the MrgprX4 antagonist compound is selected from the group comprising an antibody or fragment thereof, a binding protein, a polypeptide, a small molecule, a nucleic acid, or any combination thereof.

23. A kit comprising 1) a pharmaceutical composition of claim 18 and 2) written instructions for treating melanoma.

24-26. (canceled)
